(19) United States
(12) Patent Application Publication
(10) Pub. No.: US 2024/0374328 A1
(43) Pub. Date: Nov. 14, 2024

(54) SYSTEM AND METHOD OF OPERATING SURGICAL ROBOTIC SYSTEMS WITH ACCESS PORTS

(71) Applicant: Covidien LP, Mansfield, MA (US)

(72) Inventors: Andrew W. Zeccola, Allston, MA (US); Jared N. Farlow, Los Angeles, CA (US); Jaimeen V. Kapadia, Cambridge, MA (US); Paul M. Loschak, Somerville, MA (US); Alok Agrawal, New Haven, CT (US); Gregory A. Dierksen, Arlington, MA (US); Sanjay Jonnavithula, New York, NY (US); Colin H. Murphy, Cambridge, MA (US)

(21) Appl. No.: 18/293,082

(22) PCT Filed: Aug. 18, 2022
(86) PCT No.: PCT/IB2022/057722

§ 371 Date: Jan. 29, 2024

Related U.S. Application Data
(60) Provisional application No. 63/237,550, filed on Aug. 27, 2021.

(51) Int. Cl. A61B 34/37; A61B 34/10; A61B 34/30; A61B 90/00

(52) U.S. Cl. CPC A61B 34/37; A61B 90/37; A61B 2034/108; A61B 2034/302; A61B 2090/066; A61B 2562/0261

(57) ABSTRACT

A surgical robotic system is configured to output a recommendation for use of an access port based on a determined habitus of a patient. The system includes a robotic arm including at least one joint having a sensor. The system also includes a controller configured to receive a measured torque of the joint from the sensor and determine a habitus of the patient based on the measured torque.

### BACKGROUND

Surgical robotic systems are currently being used in minimally invasive medical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body.

Certain surgical robotic systems do not support automatic identification of a type of port/trocar that is attached to each arm, so the systems are not aware of port properties such as length, which may vary from patient to patient based on a patient's habitus. Habitus is a term used to describe the body build and constitution of an individual and may be based on any one or more physical characteristics of the individual such as, for example, weight, height, bone density, and soft issue thickness.

### SUMMARY

In surgery, access ports may have varying lengths. For example, bariatric ports may be longer than the standard-length ports. Long ports feature the same parts as standard-length ports. The long ports use the same types of port seals and attach to the robot arm by the same port latch. The remote center of motion (RCM) is the same distance from the robot arm. The key difference between long ports and standard-length ports is that the long port features a few additional centimeters of port length below the RCM (e.g., the portion that extends into the patient). This disclosure proposes a surgical robotic system configured to provide a recommendation for an access port that is suitable for use with a patient based on the patient's habitus. The system may also enable user confirmation of whether an access port to be used with the surgical robotic system is suitable for use with the patient based on the patient's habitus. For example, the disclosed surgical robotic system may determine a patient's habitus as corresponding to one of a standard-sized patient or a bariatric patient. Based on the patient's determined habitus, the surgical robotic system outputs a recommendation for an access port of suitable length (e.g., standard or long) to be used for the patient. The surgical robotic system may also output a request for the user to confirm that the access port to be used with the surgical robotic system is of a suitable length for use with a patient based on the patient's determined habitus.

Provided in accordance with aspects of the present disclosure is a surgical robotic system including a robotic arm configured to support an access port inserted within a patient and a surgical instrument inserted into the access port. The robotic arm includes at least one joint having a sensor. The system also includes a surgeon console configured to receive user input and a controller. The controller is configured to receive a measured torque of the at least one joint from the sensor and compare the measured torque to a predetermined threshold. Based on the comparison between the measured torque and the predetermined threshold, the controller is configured to determine a habitus of the patient. The controller is also configured to output a recommendation for the access port based on the determined habitus of the patient.

In some aspects of the disclosure, the controller is configured to output a request for a user input confirmation that a length of the access port corresponds to the determined habitus of the patient.

In some aspects of the disclosure, the controller is configured to disable operation of the surgical instrument until the user input confirmation is received and enable operation of the surgical instrument in response to receiving the user input confirmation.

In some aspects of the disclosure, the controller is configured to output the recommendation via a display.

In some aspects of the disclosure, the sensor includes a strain gauge configured to convert a mechanical force into a sensor signal.

In some aspects of the disclosure, the controller is configured to determine the habitus of the patient based on a number of times the measured torque exceeds the predetermined threshold.

In some aspects of the disclosure, the robotic arm includes a plurality of joints and the controller is configured to receive a measured torque of each joint of the plurality of joints and calculate a mean of the measured torques received from each joint of the plurality of joints.

In some aspects of the disclosure, the controller is configured to determine the habitus of the patient based on a comparison of the mean of the measured torques to the predetermined threshold.

Also provided in accordance with aspects of the disclosure is a surgical robotic system including a display and a robotic arm configured to support an access port inserted within a patient and an instrument having an end effector inserted into the access port. The robotic arm includes at least one joint having a sensor. The system also includes a surgeon console configured to receive user input and a controller. The controller is configured to receive a measured torque of the joint from the at least one sensor and output, on the display, a recommendation for the access port based on the received measured torque.

In some aspects of the disclosure, the controller is configured to output, on the display, a request for a user input confirmation of a length of the access port based on the received measured torque.

In some aspects of the disclosure, the controller is configured to disable operation of the surgical instrument until the user input confirmation is received and enable operation of the surgical instrument in response to receiving the user input confirmation.

In some aspects of the disclosure, the controller is configured to output the confirmation request via a display.

In some aspects of the disclosure, the sensor includes a strain gauge configured to convert a mechanical force into a sensor signal.

In some aspects of the disclosure, the controller is configured to determine a habitus of the patient based on a number of times the measured torque exceeds the predetermined threshold.

In some aspects of the disclosure, the robotic arm includes a plurality of joints and the controller is configured to receive a measured torque of each joint of the plurality of joints and calculate a mean of the measured torques received from each joint of the plurality of joints.

In some aspects of the disclosure, the controller is configured to determine a habitus of the patient based on a comparison of the mean of the measured torques to the predetermined threshold.

Also provided in accordance with the disclosure is a method for controlling a surgical robot. The method includes outputting a drive command at a main controller to actuate a robotic arm having at least one joint and measuring torque of the at least one joint using a sensor during actuation of the robotic arm. The method also includes receiving the measured torque of the joint from the sensor and comparing the measured torque to a predetermined threshold. The method also includes determining a habitus of a patient based on the comparison and outputting a recommendation for an access port to be used with the robotic arm based on the determined habitus of the patient.

In some aspects of the disclosure, the method also includes outputting a request for a user input confirmation that a length of an access port supported by the robotic arm corresponds to the determined habitus of the patient.

In some aspects of the disclosure, the method also includes disabling operation of a surgical instrument supported by the robotic arm until the user input confirmation is received and enabling operation of the surgical instrument in response to receiving the user input confirmation.

In some aspects of the disclosure, the method also includes measuring torque of a plurality of joints, receiving the measured torques of the plurality of joints, and calculating a mean of the received measured torques.

In some aspects of the disclosure, the method also includes determining the habitus of the patient based on a comparison of the mean to the predetermined threshold.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms according to an embodiment of the present disclosure;

FIG. 2 is a perspective view of a surgical robotic arm of the surgical robotic system of FIG. 1 according to an embodiment of the disclosure;

FIG. 3 is a perspective view of a setup arm with the surgical robotic arm of the surgical robotic system of FIG. 1 according to an embodiment of the disclosure;

FIG. 4 is a schematic diagram of a computer architecture of the surgical robotic system of FIG. 1 according to an embodiment of the disclosure;

FIG. 5 is a cross-sectional view of an actuator of the surgical robotic arm of FIG. 2 according to the disclosure;

FIG. 6 is a side view of access ports of different lengths according to the disclosure; and

FIG. 7 is a flow chart of an example approach for controlling the surgical robotic system of FIG. 1 according to an embodiment of the disclosure.

### DETAILED DESCRIPTION

Embodiments of the disclosed surgical robotic system are described in detail with reference to the drawings, in which like reference numerals designate identical or corresponding elements in each of the several views. As used herein the term “distal” refers to the portion of the surgical robotic system and/or the surgical instrument coupled thereto that is closer to the patient, while the term “proximal” refers to the portion that is farther from the patient.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller, or on a user device, including, for example, a mobile device, a personal computer, or a server system.

As will be described in detail below, this disclosure is directed to a surgical robotic system, which includes a surgical console, a control tower, and one or more movable carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

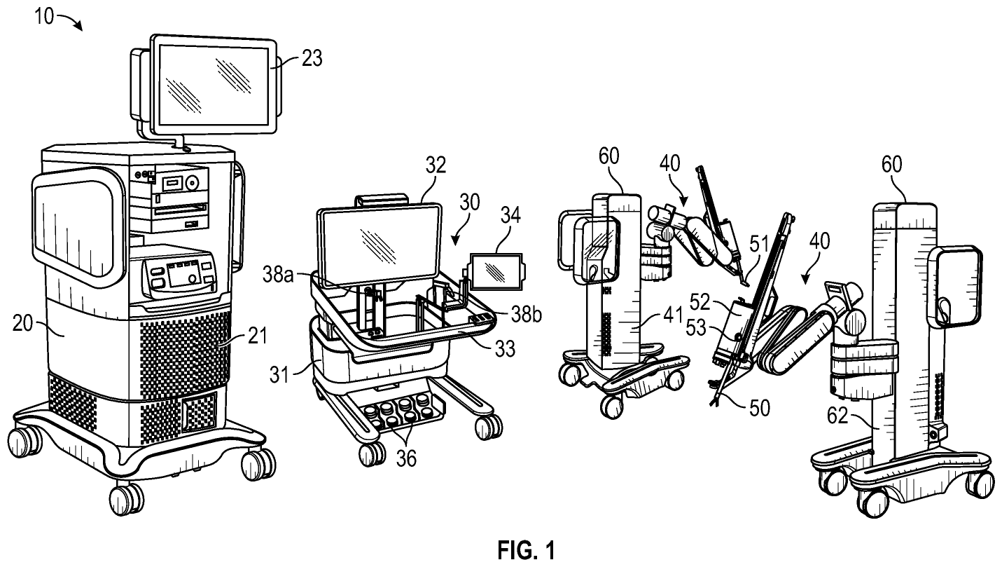

With reference to FIG. 1, a surgical robotic system 10 includes a control tower 20, which is connected to all of the components of the surgical robotic system 10 including a surgical console 30 and one or more robotic arms 40. Each of the robotic arms 40 includes a surgical instrument 50 removably coupled thereto. Each of the robotic arms 40 is also coupled to a movable cart 60.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include a camera 51 configured to capture video of the surgical site. The surgical console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgical console 30 also includes a plurality of user interface devices, such as foot pedals 36 and a pair of hand controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the hand controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

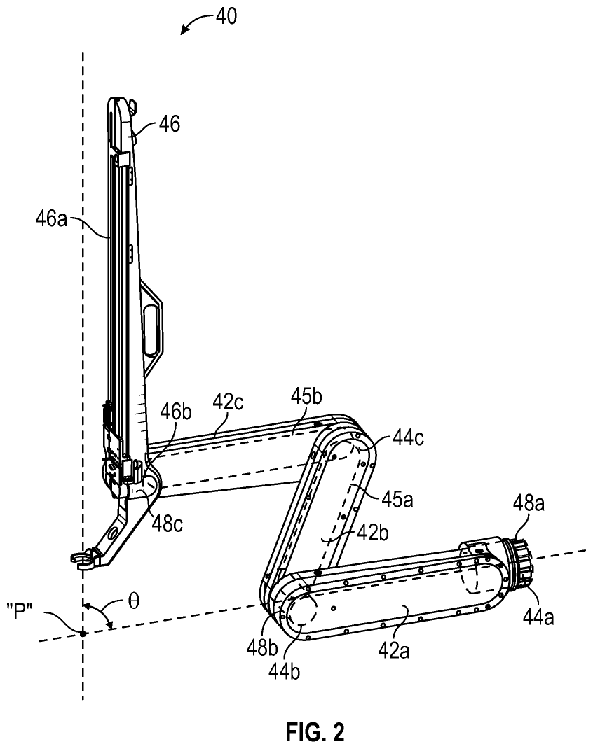

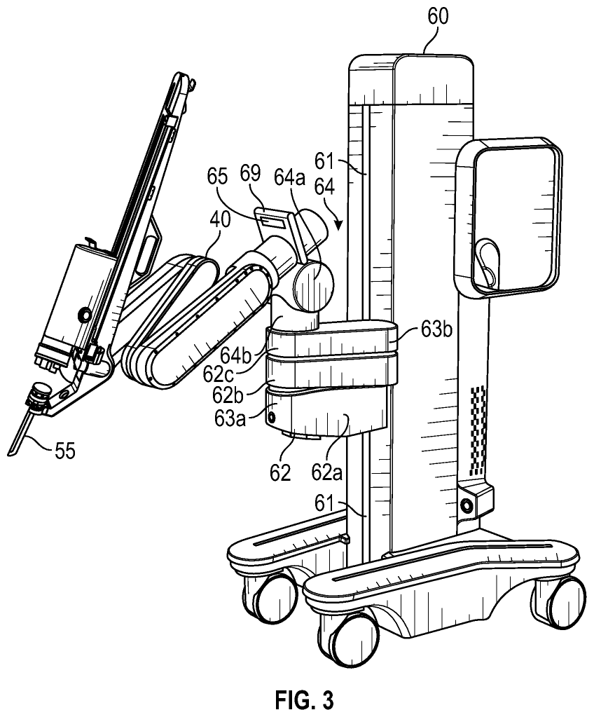

With reference to FIG. 2, each of the robotic arms 40 may include a plurality of links 42a, 42b, 42c. Links 42a and 42b are interconnected at a joint 44b and links 42b and 42c are interconnected at a joint 44c. The robotic arm 40 is secured to the movable cart 60 by a joint 44a, which defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

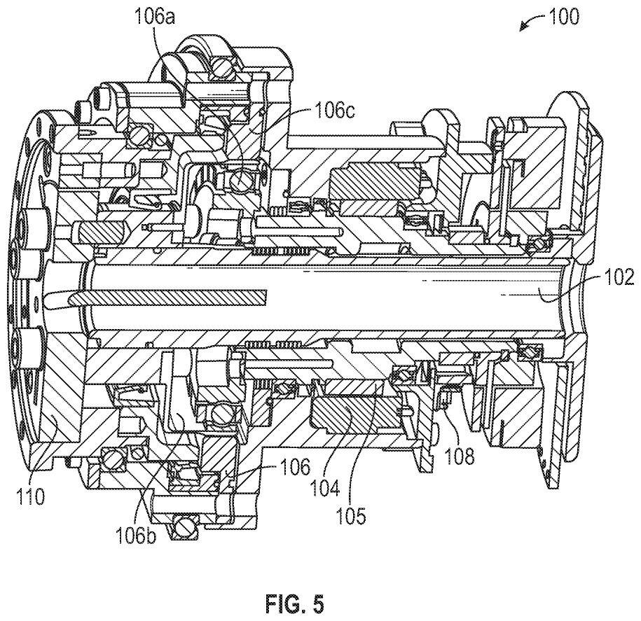

With reference to FIG. 2, the robotic arm 40 also includes a holder 46 defining a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., an end effector 200 shown in FIG. 5) of the surgical instrument 50. The holder 46 includes a sliding mechanism 46a, which is configured to move the IDU 52 along the second longitudinal axis defined by the holder 46. The holder 46 also includes a joint 44d about which the holder 46 rotates relative to the link 42c. More specifically, the joint 44d includes an actuator 48c configured to drive joint 44d to rotate the holder 46 about the second longitudinal axis defined by the holder 46. During endoscopic procedures, the instrument 50 may be inserted through an endoscopic port 55 (FIG. 3) held by the holder 46.

The robotic arm 40 also includes a plurality of manual control buttons 53 (FIG. 1) disposed on the IDU 52 and the setup arm 62, which may be used in a manual mode. The user may press one or more of the buttons 53 to move the component associated with the button 53.

The joints 44a and 44b include an actuator 48a and 48b, respectively, configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In some embodiments, some or all of the joints 44a, 44b, 44c may include an actuator to obviate the need for mechanical linkages.

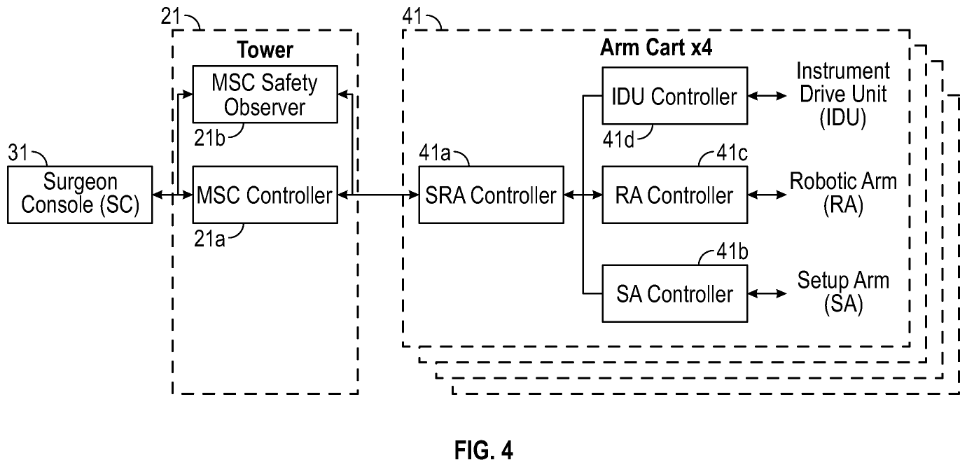

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the hand controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgical console 30 to provide haptic feedback through the hand controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the hand controller controlling the robotic arm 40, e.g., the hand controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the hand controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the hand controller 38a is then scaled by a scaling function executed by the controller 21a. In embodiments, the coordinate position is scaled down and the orientation is scaled up by the scaling function. In addition, the controller 21a also executes a clutching function, which disengages the hand controller 38a from the robotic arm 40. In particular, the controller 21a stops transmitting movement commands from the hand controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the hand controller 38a and is then passed by an inverse kinematics function executed by the controller 21a. The inverse kinematics function calculates angles for the joints 44a, 44b, 44c of the robotic arm 40 that achieve the scaled and adjusted pose input by the hand controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

The robotic arm controller 41c is also configured to estimate torque imparted on the joints 44a and 44b by the rigid link structure of the robotic arm 40, namely, the links 42a, 42b, 42c. Joint 44a houses actuator 48a and joint 44b houses actuator 48b. High torque may be used to move the robotic arm 40 due to the heavy weight of the robotic arm 40. However, the torque may need to be adjusted to prevent damage or injury. This is particularly useful for limiting torque during collisions of the robotic arm 40 with external objects, such as other robotic arms, patient, staff, operating room equipment, etc.

In order to determine the effect of external torque on the robotic arm 40 the robotic arm controller 41c initially calculates frictional losses, gravitational forces, inertia, and then determines the effects of external torque. Once the external torque is calculated, the robotic arm controller 41c determines whether the environmental forces exceed a predetermined threshold which is indicative of collisions with external objects and takes precautionary action, such as terminating movement in the direction in which collision was detected, slowing down, and/or reversing movement (e.g., moving in an opposite direction) for a predetermined distance.

The sensor measurements and calculations based thereon are described below with respect to FIG. 5, which shows an integrated joint module 100. The integrated joint module 100 is an example actuator that may be used as any one or more of the actuators 48a, 48b, 48c. The integrated joint module 100 includes a shaft 102, which acts as a support structure for the other components of the integrated joint module 100, namely, a motor 104 and a harmonic gearbox 106. The motor 104 may be any electric motor, which may be powered by AC or DC energy, such as a brushed motor, a brushless motor, a stepper motor, and the like. The motor 104 is coupled to the harmonic gearbox 106, which may be a harmonic drive gear configured to provide a large reduction ratio with approximately zero backlash, high torque capability, and high efficiency. The harmonic gearbox 106 may include concentric input and output shafts (not shown) and may include a wave generator 106a, disposed within a flexspline 106b having an outer geared surface, which is in turn, disposed within a circular spline 106c having an inner geared surface. As the motor 104 drives the wave generator 106a, the flexspline 106b, which may be formed from an elastic material, such as stainless steel, is also rotated. The flexspline 106b has fewer teeth than the circular spline 106c, therefore for every full rotation of the wave generator 106a, the flexspline 106b rotates less than a full rotation, which reduces the output speed. The harmonic gearbox 106 is in turn coupled to one of the belts 45a or 45b.

The integrated joint module 100 also includes a sensor suite for monitoring the performance of the integrated joint module 100 to provide for feedback and control thereof. In particular, the integrated joint module 100 includes an encoder 108 coupled to the motor 104. The encoder 108 may be any device that provides a sensor signal indicative of the number of rotations of the motor 104, such as a mechanical encoder or an optical encoder. The motor 104 may also include other sensors, such as a current sensor configured to measure the current draw of the motor 104, a motor torque sensor 105 for measuring motor torque, and the like. The number of rotations may be used to determine the speed and/or position control of individual joints 44a, 44b, 44c, 46b. Parameters that are measured and/or determined by the encoder 108 may include speed, distance, revolutions per minute, position, and the like. The integrated joint module 100 further includes a joint torque sensor 110 that may be any force or strain sensor including one or more strain gauges and/or load cells configured to convert mechanical forces (e.g., strain) into a sensor signal indicative of the torque imparted by the harmonic gearbox 106. The sensor signals from the encoder 108 and the joint torque sensor 110 are transmitted to the computer 41, which then controls the speed, angle, and/or position of each of the joints 44a, 44b, 44c of the robotic arm 40 based on the sensor signals. In embodiments, additional position sensors may also be used to determine movement and orientation of the robotic arm 40 and the setup arm 62. Suitable sensors include, but are not limited to, potentiometers coupled to movable components and configured to detect travel distances, Hall Effect sensors, accelerometers, and gyroscopes.

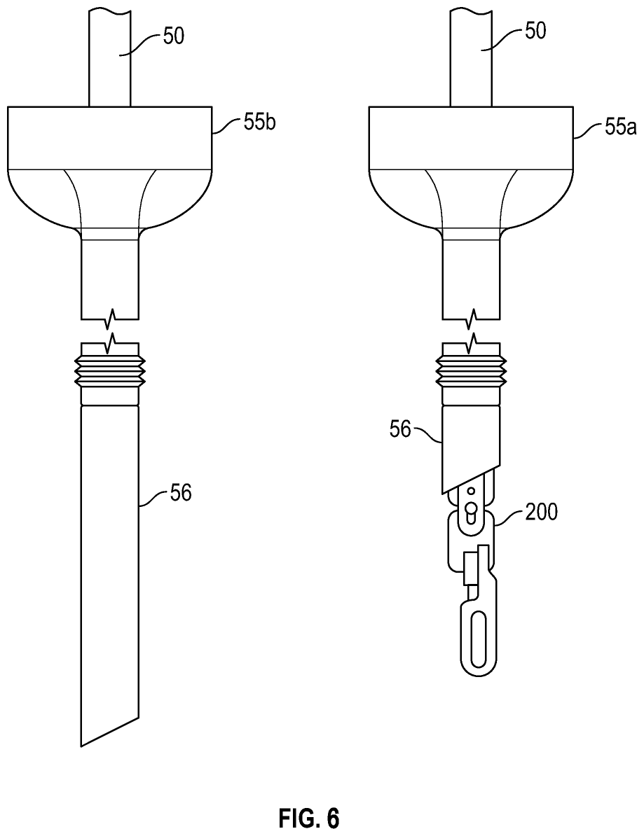

With reference to FIG. 6, during surgery, ports of different lengths may be used, in particular, long ports 55b may be used for bariatric surgery, which are longer than standard ports 55a. Use of longer ports affects the functionality of the system 100 during instrument exchange and position control of the instrument 50 inside the port 55. More specifically, instrument exchange requires the wristed instrument to be straightened and closed during extraction before the first proximal joint reaches the bottom of the port.

During use of the instrument 50, the instrument 50 is inserted into a longitudinal tube 56 of the port 55 by advancing the instrument 50 and the IDU 52 along the sliding mechanism 46a. The instrument 50, and in particular an end effector 200 is advanced to a desired depth. The distance to which the instrument 50, and in particular, the end effector 200 has been advanced is continuously tracked by the IDU controller 41d and other controllers (e.g., controller 21a). It has been observed that the effort necessary to manipulate the instrument 50 within a patient during surgery varies with patient habitus. For example, relatively more effort may be necessary to manipulate the instrument 50 within a bariatric patient with thick body walls, as compared to a smaller (e.g., standard or petite) patient where relatively less effort may be necessary to manipulate the instrument 50 within the patient. As such, by observing torque data at one or more joints of the robotic arm 40 (e.g., joints 44a, 44b, and 46b) during manipulation of the instrument 50 within the patient (e.g., during a surgical procedure or during calibration of the instrument 50), a habitus (e.g., bariatric, standard, petite, etc.) of the patient can be determined. Various metrics can be applied to differentiate between patients based on habitus. For example, a mean torque value and/or a standard deviation from the mean torque value may be determined based on torque data obtained from any one or more of joints 44a, 44b, and 46b and compared to a predetermined threshold, which may be empirically obtained based on testing of a population of patients of varying habitus (e.g., bariatric, standard, petite, etc.) and/or mechanical properties of the instrument 50. Additionally or alternatively, torque data (e.g., mean torque value) may be compared to the predetermined threshold and, depending on a number of times the torque data exceeds the predetermined threshold, the habitus of the patient may be determined. Another metric that may be considered is determining a distribution of torque across any combination of the joints 44a, 44b, and 46b and comparing the determined distribution to corresponding distribution data empirically obtained based on testing of a population of patients of varying habitus. Other inputs for differentiating between patients based on habitus may be considered, examples of which include but are not limited to relative location of carts (e.g., cart 60) with respect to the bed on which the patient is lying, orientation of the setup arm 62, depth of insertion of the instrument 50 through the access port 55, range of articulation angles of the instrument 50, and/or correlation between joint angles and joint forces.

Empirical testing of a population of patients may include determining for each patient of the test population a maximum torque value at each of joints 44a, 44b, and 46b during a surgical procedure.

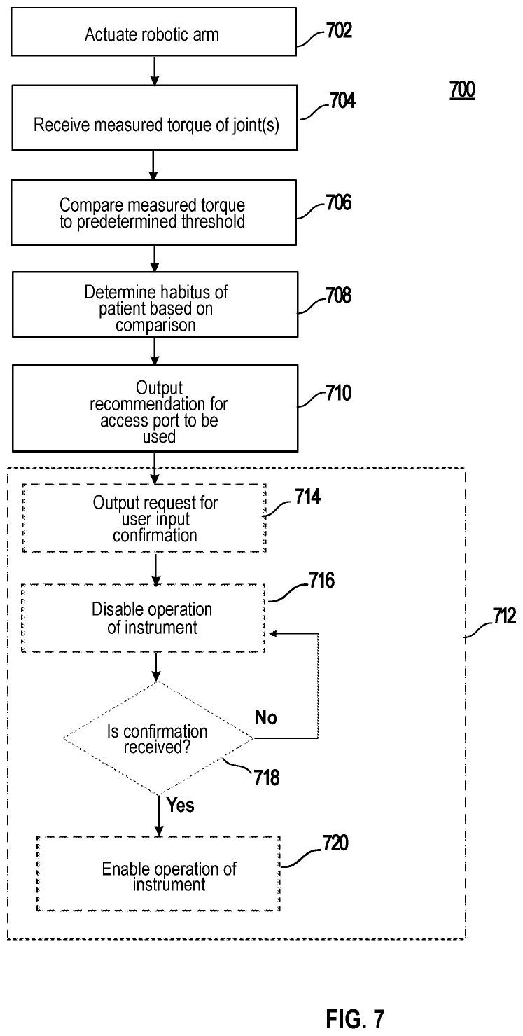

With reference to FIG. 7, an example approach 700 for determining a habitus of a patient and outputting a recommendation for an access port to be used is shown. In FIG. 7, various blocks are illustrated as optional (e.g., block 712 denoted by dashed lines). However, this disclosure contemplates blocks in which one or more blocks of the example approach 700 are optional, omitted, and/or alternatively performed according to various aspects. Further, one or more operations of the example approach 700 may be transposed and/or contemporaneously performed.

Initially at block 702, a drive command is output to actuate the robotic arm 40. At block 704, measured torque from one or more joints (e.g., 44a, 44b, 46b) of robotic arm 40 is received and, at block 706, the measured torque is compared to a predetermined threshold. At block 708, a habitus of the patient is determined based on the comparison between the received measured torque and the predetermined threshold. At block 710, a recommendation for an access port to be used is output based on the determined habitus of the patient. The recommendation may be output on any of the displays 23, 32, and 34 of the control tower 20 and the surgeon console 30, respectively. For example, a patient's habitus may be determined as corresponding to a large or bariatric patient, which may cause the system to output a recommendation for use of a long access port (e.g., long port 55b). By way of another example, a patient's habitus may be determined as corresponding to a standard-sized patient, which may cause the system to output a recommendation for use of a standard-length access port (e.g., standard port 55a).

Optionally, the example approach 700 may proceed to block 712, which includes block 714 at which a request is output for a user input confirmation that an access port to be used corresponds to the determined habitus of the patient. The confirmation request may be for a user to confirm that an access port to be used with the surgical robotic system 10 (e.g., an access port corresponding to the recommendation output at block 710) is of a suitable length for use with a patient having the patient's determined habitus. The confirmation request may be output on any of the displays 23, 32, and 34 of the control tower 20 and the surgeon console 30, respectively. Confirmation may be received by touching a corresponding button, e.g., “YES”, displayed on any of the displays 23, 32, and 34. In some embodiments, the surgeon may input confirmation by touching a sensor (not shown) at the surgeon console 30 using any suitable gesture, such as a double tap. Furthermore, confirmation may be received by pressing one of the foot pedals 36 of the surgeon console 30. At block 716, operation of the instrument 50 is disabled by the controller 21a until the user input confirmation is received. Operation of the instrument 50 remains disabled (block 716) until the user input confirmation is received (block 718). In response to the user input confirmation being received, the controller 21a enables operation of the instrument (block 720).

It will be understood that various modifications may be made to the embodiments disclosed herein. In embodiments, the sensors may be disposed on any suitable portion of the robotic arm. Therefore, the above description should not be construed as limiting, but merely as exemplifications of various embodiments. Those skilled in the art will envision other modifications within the scope and spirit of the claims appended thereto.

What is claimed is:

1. A surgical robotic system, comprising:
a robotic arm configured to support an access port inserted within a patient and a surgical instrument inserted into the access port, the robotic arm including at least one joint having a sensor;
a surgeon console configured to receive user input; and
a controller configured to:
receive a measured torque of the at least one joint from the sensor;
compare the measured torque to a predetermined threshold;
determine a habitus of the patient based on the comparison; and
output a recommendation for the access port based on the determined habitus of the patient.

2. The surgical robotic system according to claim 1, wherein the controller is configured to output a request for a user input confirmation that a length of the access port corresponds to the determined habitus of the patient.

3. The surgical robotic system according to claim 2, wherein the controller is configured to:
disable operation of the surgical instrument until the user input confirmation is received; and
enable operation of the surgical instrument in response to receiving the user input confirmation.

4. The surgical robotic system according to claim 1, wherein the controller is configured to output the recommendation via a display.

5. The surgical robotic system according to claim 1, wherein the sensor includes a strain gauge configured to convert a mechanical force into a sensor signal.

6. The surgical robotic system according to claim 1, wherein the controller is configured to determine the habitus of the patient based on a number of times the measured torque exceeds the predetermined threshold.

7. The surgical robotic system according to claim 1, wherein the robotic arm includes a plurality of joints and the controller is configured to:
receive a measured torque of each joint of the plurality of joints; and
calculate a mean of the measured torques received from each joint of the plurality of joints.

8. The surgical robotic system according to claim 7, wherein the controller is configured to determine the habitus of the patient based on a comparison of the mean of the measured torques to the predetermined threshold.

9. A surgical robotic system, comprising:
a display;
a robotic arm configured to support an access port inserted within a patient and an instrument having an end effector inserted into the access port, the robotic arm including at least one joint having a sensor;
a surgeon console configured to receive user input; and
a controller configured to:
receive a measured torque of the joint from the at least one sensor; and
output, on the display, a recommendation for the access port based on the received measured torque.

10. The surgical robotic system according to claim 9, wherein the controller is configured to output, on the display, a request for a user input confirmation of a length of the access port based on the received measured torque.

11. The surgical robotic system according to claim 10, wherein the controller is configured to:
disable operation of the surgical instrument until the user input confirmation is received; and
enable operation of the surgical instrument in response to receiving the user input confirmation.

12. The surgical robotic system according to claim 9, wherein the sensor includes a strain gauge configured to convert a mechanical force into a sensor signal.

13. The surgical robotic system according to claim 9, wherein the controller is configured to determine a habitus of the patient based on a number of times the measured torque exceeds the predetermined threshold.

14. The surgical robotic system according to claim 9, wherein the robotic arm includes a plurality of joints and the controller is configured to:
receive a measured torque of each joint of the plurality of joints; and
calculate a mean of the measured torques received from each joint of the plurality of joints.

15. The surgical robotic system according to claim 14, wherein the controller is configured to determine a habitus of the patient based on a comparison of the mean to the predetermined threshold.

16. A method for controlling a surgical robot, the method comprising:
outputting a drive command at a main controller to actuate a robotic arm having at least one joint;
measuring torque of the at least one joint using a sensor during actuation of the robotic arm;
receiving the measured torque of the joint from the sensor;
comparing the measured torque to a predetermined threshold;
determining a habitus of a patient based on the comparison; and
outputting a recommendation for an access port to be used with the robotic arm based on the determined habitus of the patient.

17. The method according to claim 16, further comprising outputting a request for a user input confirmation that a length of an access port supported by the robotic arm corresponds to the determined habitus of the patient.

18. The method according to claim 17, further comprising:
disabling operation of a surgical instrument supported by the robotic arm until the user input confirmation is received; and
enabling operation of the surgical instrument in response to receiving the user input confirmation.

19. The method according to claim 16, further comprising:
measuring torque of a plurality of joints;
receiving the measured torques of the plurality of joints; and
calculating a mean of the received measured torques.

20. The method according to claim 19, further comprising determining the habitus of the patient based on a comparison of the mean of the measured torques to the predetermined threshold.
